(19) United States
(12) Patent Application Publication
(10) Pub. No.: US 2004/0063641 A1
(43) Pub. Date: Apr. 1, 2004

(54) Use of growth hormone or a growth hormone secretagogue for promoting bone formation

(76) Inventors: Michael Raschke, Berlin (DE); Henning Windhagen, Hannover (DE)

Correspondence Address: Reza Green, Esq., Novo Nordisk, Pharmaceuticals, Inc., 100 College Road West, Princeton, NJ, 08540, US

(21) Appl. No.: 10/669,538

(22) Filed: Sep. 24, 2003

(30) Foreign Application Priority Data
Nov. 5, 1996 (DK) 1239/96

(51) Int. Cl. A61K 38/05; C07K 5/06

(52) U.S. Cl. 514/18; 530/331

(57) ABSTRACT

The invention provides a method of enhancing the healing of bone fractures in an animal subjected to distraction osteogenesis, the method comprising administering, to the animal in need thereof, a growth hormone or a growth hormone secretagogue in an amount sufficient to provide bone formation simultaneous with the distraction procedure.

The invention further provides a method for treatment of patients suffering from fractured bones by distractive osteogenesis, which method comprises administering an effective amount of a growth hormone or a growth hormone secretagogue to a patient in need of such a treatment in conjunction with the distraction procedure, as well the use of a growth hormone or a growth hormone secretagogue for the manufacture of a medicament for the enhancement of healing of bone fractures in distractive osteogenesis,

### CROSS-REFERENCE TO RELATED APPLICATIONS

This application claims priority under 35 U.S.C. 119 of Danish application no. PA 1239/96 filed on Nov. 5, 1996, and U.S. application No. 60/035,657 filed on Dec. 23, 1996 and claims priority under 35 U.S.C. 120 of U.S. application Ser. No. 08/963,388 filed on Nov. 3, 1997 and of U.S. application Ser. No. 10/015,342 filed Dec. 13, 2001, the contents of which are fully incorporated herein by reference.

### FIELD OF THE INVENTION

The present invention relates to the use of a growth hormone or a growth hormone secretagogue for promoting bone formation in an animal simultaneous with callus distraction.

The present invention further relates to a method of enhancing the healing of bone fractures in an animal subjected to distraction osteogenesis by administering, to the animal in need thereof, a growth hormone or a growth hormone secretagogue in an amount sufficient to provide bone formation simultaneous with the distraction procedure.

The invention further relates to the uses of a growth hormone or a growth hormone secretagogue for the manufacture of a medicament for the promotion of bone formation simultaneous with callus distraction and manufacture of a medicament for the enhancement of healing of bone fractures in distraction osteogenesis,

The invention further relates to a method of treating patients suffering from fractures, post-traumatic and idiopathic deformities of extremities by distraction osteogenesis, which method comprises administering an effective amount of a growth hormone or a growth hormone secretagogue to a patient in need of such a treatment in conjunction with the distraction procedure.

### BACKGROUND OF THE INVENTION

Bone formation and healing of fractures are depending on basal biological processes which seem to be related. These processes, which have not yet been fully understood, are being studied intensively, partly to understand the biological correlations, and partly to develop “biological tools” enabling influencing or stimulating the processes of healing.

The sequence of biological responses in bone healing in its broad sense may be outlined as follows:

A trauma elicits release of bone-derived growth factors from the bone matrix as well as other local growth factors from the surrounding tissue and the blood. These factors of which a number is known elicits 1) an increased metabolism in the area, 2) changes of the secretion of superior-hormones, and 3) a specific reaction leading to differentiation of primitive cells to form bone cells and proliferation of these. This specific reaction depends on the interaction between several polypeptide growth factors being dependant on hormones.

Distraction osteogenesis (callus distraction) is a widely accepted method for limb lengthening and bone transport (see, for example, Aronson et al., Clinical Orthopaedics and Related Research (1989 April) 241:106-116, Paley et al., Clinical Orthopaedics and Related Research (1989 April) 241:146-165, Aronson et al., Clinical Orthopaedics and Related Research (1989 Junel) 243:71-9). In short, it is a method for salvaging limbs with severe congenital, post-traumatic, or other acquired deformities. The method, which generally use a universal system of ring external fixators with tensioned trans-osseous wires, has been developed over the past 35 years. The semi-invasive technique have proved successful in over 300,000 patients (adults and children) treated for bone shortening and intercalary deficiency, angulatory and rotational malalignment, active infection, ischemia, joint contractures, non-unions, fractures, post-traumatic and idiopathic deformities of extremities. Bone transport involves moving a free segment of living bone to fill intercalary bone defects with vital bone. The trailing end of the transport bone segment maintains continuity with the host bone surface by distraction osteogenesis. The leading end of the transport bone segment fuses to the target bone surface by transformational osteogenesis. The small diameter of the trans-osseous wires contributes to better patient tolerance over the prolonged treatment times required for gradual distraction up to one millimetre a day. The method may also use a universal system of external fixators (ring and monolateral fixators) and intramedullary nails (Raschke et al. Clin Orthop. 1992). Typically, the bone fracture is distracted for a period of six months corresponding to a bone distraction of approximately 15 cm. No bone growth takes place during this six months period. For the next 1-1½ year no distraction is done, instead bone growth occurs.

However, disadvantages of this method are inconvenience to the patient and high costs due to a long term treatment period. Consequently, there is a strong need for treatment acceleration.

It has now surprisingly been found that a growth hormone or a growth hormone secretagogue accelerates bone regeneration and consolidation and thus greatly improves bone quality (e.g. more bone mass, greater torsional stiffness, greater mechanical stiffness) within a shorter term treatment period.

Our histological studies show that during the distraction period some ossification takes place at the peripheral aspect of the distraction zone, whereas in the centre of the distraction zone lengthening can still be performed. Thus an acceleration of the distraction period and the consolidation period can be seen.

When a fracture is healing, it is important that the bone is not substantially loaded until the healing process has developed sufficiently for the fracture to carry the load. The strength of the healed bone may be measured by loading the bone moderately and measuring its response to the load. Methods and devices for measuring the mechanical stiffness of a healing fractured bone are, for example, described in U.S. Pat. No. 5,339,533 and EP 0 117 859. The torsional stiffness of a healing fracture may, for example, be measured by a method where the ends of the bones at each side of the fracture are fixed to respective external holders by which they are rotated relative to each other mainly about the axis of the bone and the rotational moment necessary to perform said rotation is measured as a function or the angle of rotation.

### BACKGROUND ART

Several publications relate to the effect of growth hormone on fracture healing in rats:

Ashton et al. (Br. J. Exp. Pathol. 64:479, 1983) and Tylkowski et al. (Clin. Orthop. 115:274, 1976) disclose the healing of rat tibiae by administering growth hormone, the tibiae healing with increased strength.

Jørgensen et al. (Calcified Tissue, 44, 1989, abstract D20) describes the injection of rats with hGH. The mechanical strength of femur and tibia was measured. The maximum stiffness of the fracture was increased.

Bak et al. (Bone 11:233, 1990) describes the effect of hGH on the biomechanical properties of healing rat-tibial fractures. After 40 days of healing, the maximum stiffness of the fractures had increased.

Nielsen et al. (Acta Orthop Scand 1991, 62(3):244-247) describes the promoting effect of growth hormone on tibial fractures in rats. It is concluded that GH stimulates longitudinal bone growth by stimulating cell differentiation in the geminal zone of the growth plate. The maximum stiffness of the fractures increased in the rats injected with growth hormone for two or three weeks.

Mosekilde et al. (Bone and Mineral, The XIth International Conference on Calcium Regulating Hormones, Florence, Italy, Apr. 24-29, 1992, abstract No. 504) describes the long-term effect of growth hormone on healing rat tibial fractures. The results revealed an initial stimulatory effect of GH on callus formation, but the callus was loosely structured with significantly lower trabecular bone volume as that of non-treated rats.

Carpenter et al. (Journal of Bone and Joint Surgery, 74-A(3):359, 1992) describes the failure of growth hormone to alter the biomechanics of fracture-healing in rabbits. According to this study, administration of growth hormone had no effect on fracture-healing.

Mosekilde et al. (Bone 14:19-27, 1993) describes the effects of growth hormone on fracture healing in rats. On the basis of this study, it was concluded that although there is an initially stimulatory effect of growth hormone on callus formation, the callus formed during growth hormone treatment is abnormal with an extreme loose structure, and modelling and remodelling of this callus are delayed. It seems that the bone marrow cells grow at the expense of the mineralised callus tissue, or that the normal architecture of the callus tissue is disrupted.

Castillo et al. (Hormone Research, 33^{rd} Annual Meeting of the European Society for Paediatric Endocrinology (ESPE), Maastricht, Jun. 22-25, 1994, abstract No. 360) describes the stimulating effect of human growth hormone on fracture healing in rats. Recombinant hGH therapy accelerates healing of femur fractures in rats at 12 and 18 days post-fracture. The hGH probably acts on skeletal tissues directly by stimulating stem cells and indirectly by stimulating the production of local IGF-1.

Buonomo et al. (abstract No. P1-576 from Program & Abstracts (Vol. I: June 12-13), 10^{th} International Congress of Endocrinology, Jun. 12-13, 1996, San Francisco, USA) describes the metabolic effect of canine somatotropin(cST) on bone growth factors and fracture healing in dogs. Dogs, treated with cST, showed a 3- and 5-fold increase of strength and stiffness, respectively, of the healing fracture as compared to that of non-treated dogs.

However, no distraction of bone fractures were done in any of these studies.

The present invention relates to the enhanced healing of bone fractures during distraction osteogenesis when administering a growth hormone or a growth hormone secretagogue. When distracting the bone fractures day by day (up to two millimetres a day in pigs), no healing would be expected during the distraction period. Surprisingly, it has now been shown that a growth hormone or a growth hormone secretagogue stimulates bone growth during the distraction period and thus has an enhancing effect on overall bone healing resulting in a shorter term of treatment.

### SUMMARY OF THE INVENTION

It has now surprisingly been found that a growth hormone or a growth hormone secretagogue accelerates bone formation and regenerate consolidation and thus greatly improves bone quality (e.g. more bone mass, greater torsional stiffness, greater mechanical stiffness) within a shorter term treatment period.

It has now been shown that administration of growth hormone during the elongation or healing of fractured bones or bone defects will speed up the healing giving a more rapid development of a firm cohesion between the surfaces to be united.

According to one aspect, the invention relates to the use of a growth hormone or a growth hormone secretagogue for promoting bone formation in an animal simultaneous with callus distraction.

According to another aspect, the invention relates to a method of enhancing the healing of bone fractures in an animal subjected to distraction osteogenesis, the method comprising administering, to the animal in need thereof, a growth hormone or a growth hormone secretagogue in an amount sufficient to provide bone formation simultaneous with the distraction procedure.

According to a preferred aspect of the invention, the animal is a mammal, preferably a human being.

According to another preferred aspect of the invention, the growth hormone is human growth hormone.

According to another preferred aspect of the invention, the growth hormone secretagogue is chosen from a list of 5-amino-5-methyl-hex-2-enoic acid N-methyl-N-((1R)-1-(methyl-((1R)-1-(methylcarbamoyl)-2-phenylethyl)carbamoyl)-2-(naphthalen-2-yl)ethyl)amide with the formula I:

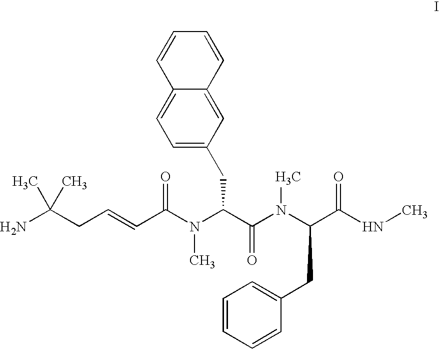

and H-Aib-His-D-2Nal-D-Phe-Lys-NH2 with the formula II:

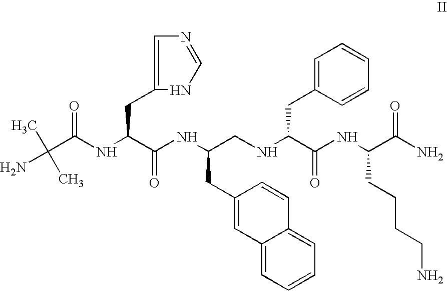

and N-[1(R)-[(1,2-Dihydro-1-methanesulfonylspiro[3H-indole-3,4′-piperidin]-1′-yl)carbonyl]-2-(phenylethyloxy)ethyl]-2-amino-2-methylpropanamide mesylate with the formula III:

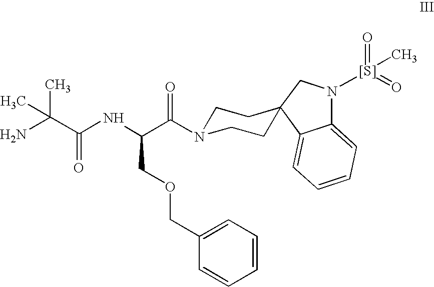

The invention further relates to the use of a growth hormone or a growth hormone secretagogue for the manufacture of a medicament for the promotion of bone formation simultaneous with callus distraction and to the use of a growth hormone or a growth hormone secretagogue for the manufacture of a medicament for the enhancement of healing bone fractures in distraction osteogenesis.

The invention further relates to a method of treating patients suffering from fractured bones, post-traumatic and idiopathic deformities of extremities by distraction osteogenesis, which method comprises administering an effective amount of a growth hormone or a growth hormone secretagogue to a patient in need of such a treatment in conjunction with the distraction procedure.

According to the invention, the route of administration may be any route which effectively transports the active compound to the appropriate or desired site of action, such as oral, nasal, vaginal, rectal, sublingual, transdermal or parenteral (e.g., intramuscular, intraperitonal, intravenous or subcutaneous injection, or implant) as well as pulmonary inhalation.

### FIGURES

FIG. 1 shows the torsional stiffness of tibia in GH-treated pigs.

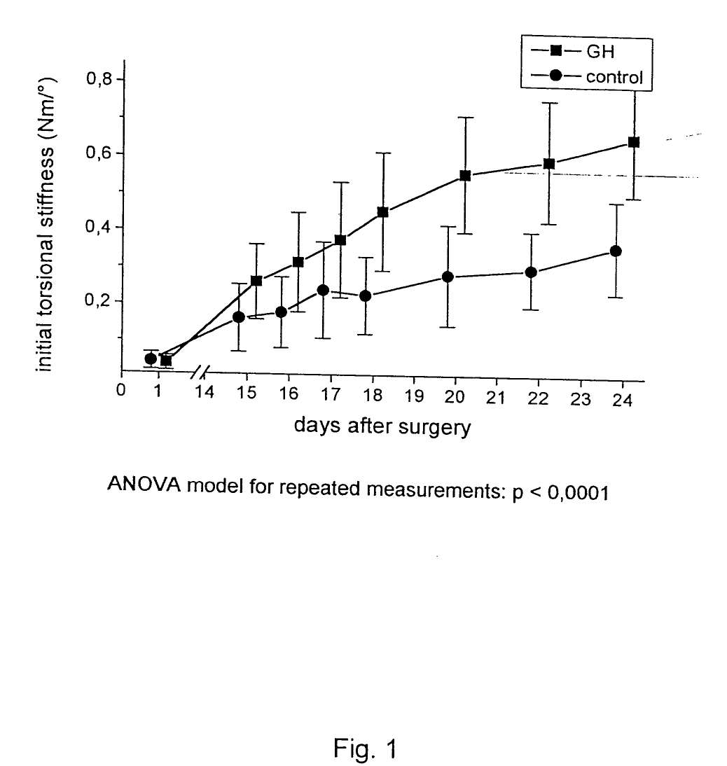

FIG. 2 shows the torsional stiffness in relation to contralateral tibia.

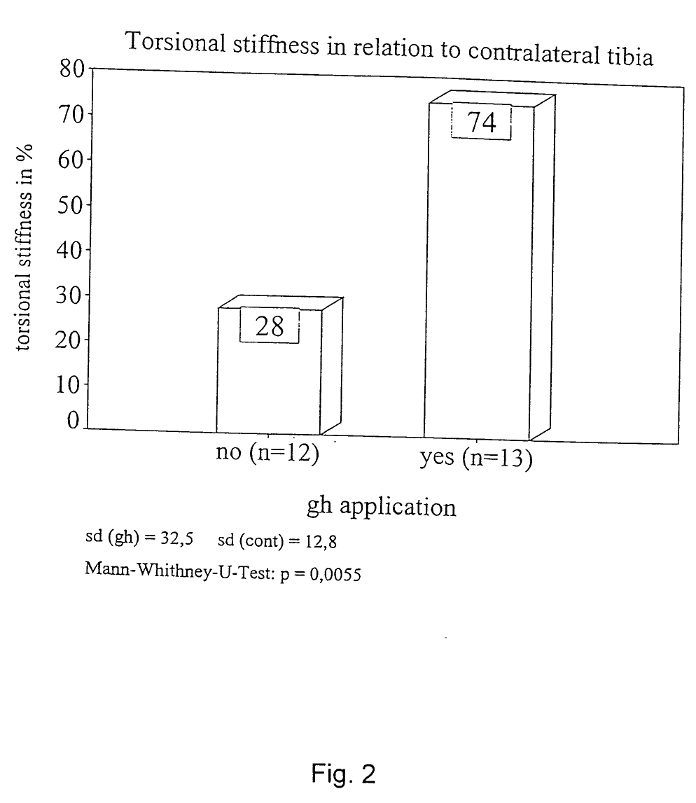

FIG. 3 shows the max. torsional moment in relation to contralateral tibia.

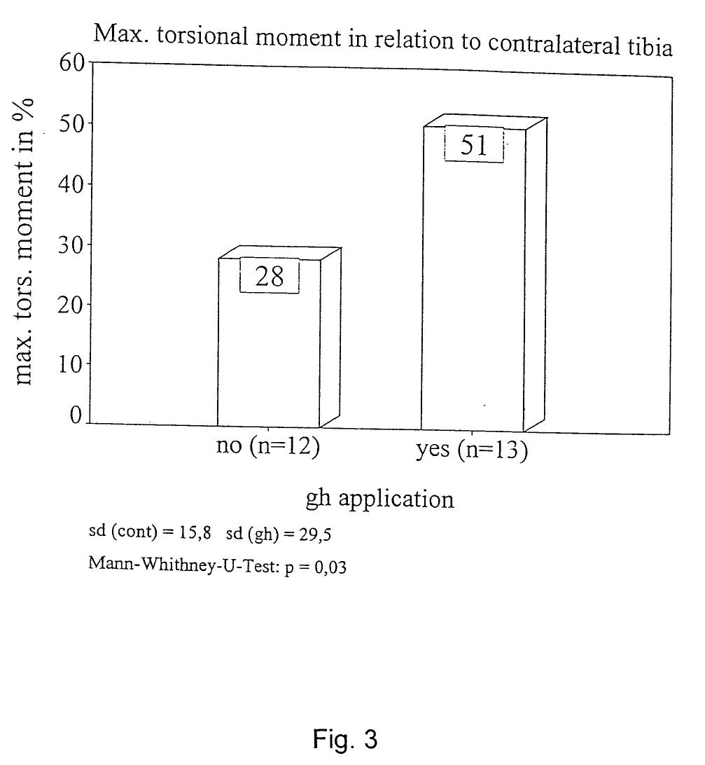

FIG. 4 shows the DIGI-measurements (digital X-ray) of tibia in GH treated pigs.

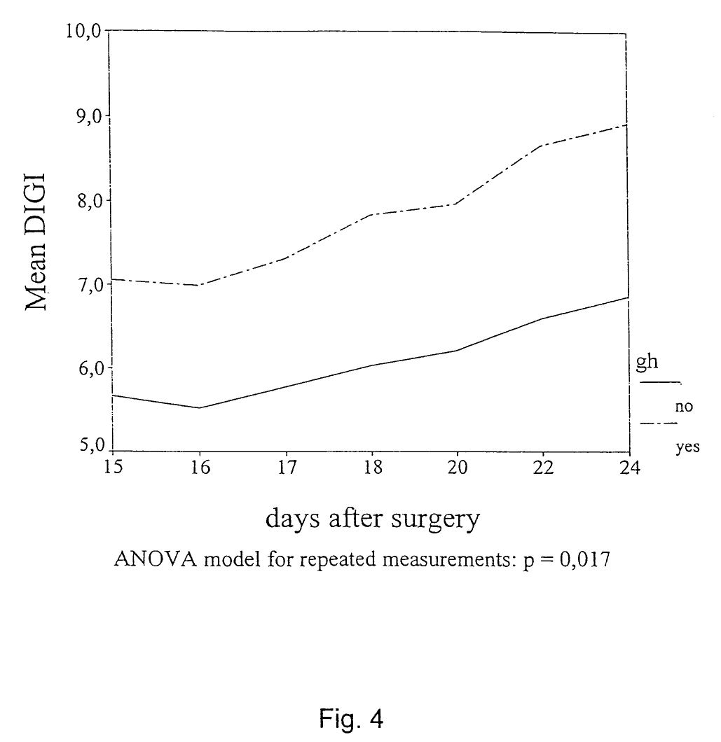

FIG. 5 shows the standard deviation (SD) on DIGI-measurements of tibia in GH treated pigs.

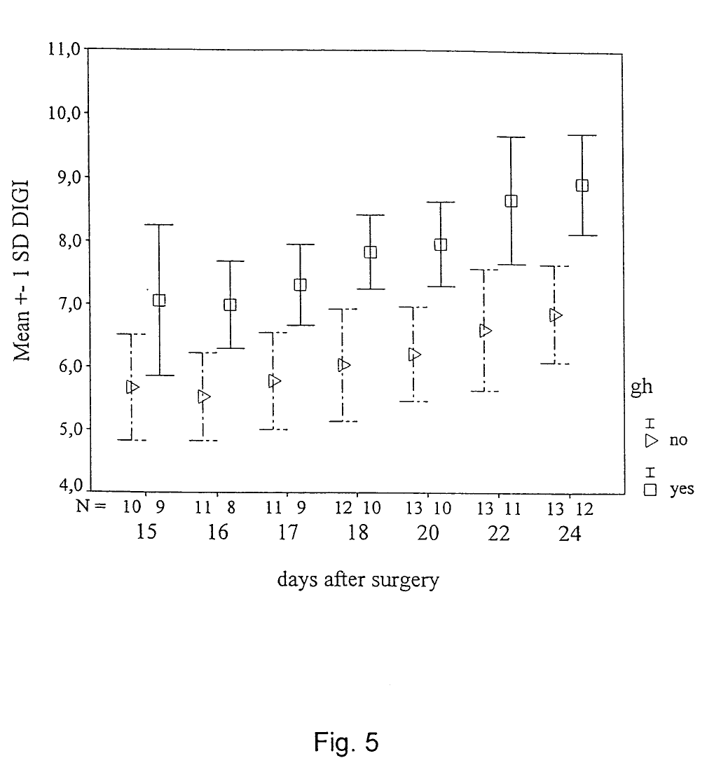

### DETAILED EXPLANATION OF THE INVENTION

Growth Hormone

Growth hormone is a hormone which stimulates growth of all tissues capable of growing. Growth hormone is released from the pituitary. The release is under tight control of a number of hormones and neurotransmitters either directly or indirectly. Growth hormone release can be stimulated by growth hormone releasing hormone (GHRH) and inhibited by somatostatin. In both cases, the hormones are released from the hypothalamus but their action is mediated primarily via specific receptors located in the pituitary. In the present context “growth hormone” may be growth hormone from any origin such as avian, bovine, equine, human, ovine, porcine, salmon, trout or tuna growth hormone, preferably bovine, human or porcine growth hormone, human growth hormone being most preferred. The growth hormone used in accordance with the invention may be native growth hormone isolated from a natural source, e.g. by extracting pituitary glands in a conventional manner, or a growth hormone produced by recombinant techniques, e.g as described in E. B. Jensen and S. Carlsen in Biotech and Bioeng. 36, 1-11 (1990). The “growth hormone derivative” may be a truncated form of growth hormone wherein one or more amino acid residues has (have) been deleted; an analogue thereof wherein one or more amino acid residues in the native molecule has (have) been substituted by another amino acid residue, preferably the residue of a naturally occurring amino acid, as long as the substitution does not have any adverse effect such as antigenicity or reduced action; or a derivative thereof, e.g. deamidated or sulfoxidated forms of the growth hormone or forms having an N- or C-terminal extension such as Met-hGH, Met-Glu-Ala-Glu-hGH or Ala-Glu-hGH. The preferred growth hormone is hGH. Growth hormone mimetics are, for example, peptides which can dimerize the GH receptor

Growth Hormone Secretagogues

Growth hormone secretagogues are compounds possessing the ability to release endogenous growth hormone in vivo. Secretagogues may, for example, be growth hormone releasing hormone (GHRH) and its analogues, growth hormone releasing factor or smaller oligo or polypeptides stimulating the release of growth hormone in vivo such as short-chain growth hormone releasing peptides (such as GHRP (2 or 6) disclosed, respectively, in WO 93/04081 and EP 83 864), or growth factors such as IGF-1 or IGF-2.

Other compounds which stimulate the release of growth hormone from the pituitary have also been described. For example arginine, L-3,4-dihydroxyphenylalanine (L-Dopa), glucagon, vasopressin, PACAP (pituitary adenylyl cyclase activating peptide), muscarinic receptor agonists and a synthethic hexapeptide, GHRP (growth hormone releasing peptide) release endogenous growth hormone either by a direct effect on the pituitary or by affecting the release of GHRH and/or somatostatin from the hypothalamus.

Compounds that increase the levels of growth hormone in mammals and which may be used according to the present invention include compounds disclosed in patents and patent applications Nos. WO 97/00894, WO 97/23508, WO 95/17422, WO 95/17423 (e.g. H-Aib-His-D-2Nal-D-Phe-Lys-NH2), WO 96/22997, WO 96/24580, WO 96/24587, WO 96/05195, and EP 18072, EP 83864, WO 89/07110, WO 89/07111, WO 89/10933, WO 88/09780, WO 83/02272, WO 91/18016, WO 92/01711, WO 93/04081, WO 96/15148, WO 96/22782, GB 2297972, GB 2298657, GB 2298647, WO 96/02530, WO 96/13265, WO 95/34311, WO 95/16675, WO 95/16692, WO 95/14666, WO 95/13069, WO 95/12598, WO 95/09633, WO 95/03290, WO 95/03289, WO 94/19367, EP 662481, WO 94/08583, WO 94/07486, WO 94/11012, WO 94/07483, WO 94/18169, WO 94/05634, and WO 92/16524. Furthermore, WO 94/13696 discloses compounds, e.g. N-[1(R)-[(1,2-Dihydro-1-methanesulfonylspiro[3H-indole-3,4′-piperidin]-1′-yl)carbonyl]-2-(phenylethyloxy)ethyl]-2-amino-2-methylpropanamide mesylate:

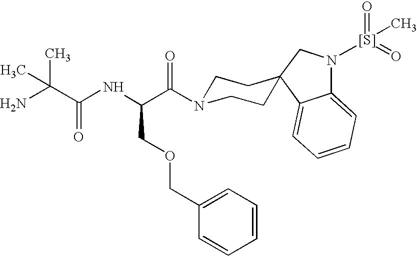

which increases the levels of growth hormone in mammals. Also, the compound 5-amino-5-methyl-hex-2-enoic acid N-methyl-N-((1R)-1-(methyl-((1R)-1-(methylcarbamoyl)-2phenylethyl)carbamoyl)-2-(naphthalen-2-yl)ethyl)amide:

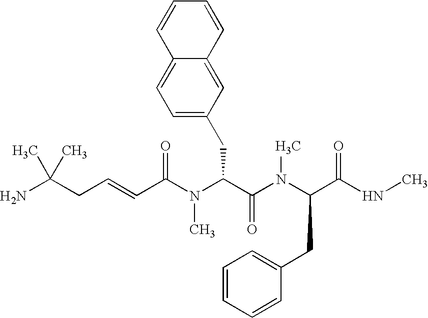

increases the levels of growth hormone in mammals. The above compounds may both be used according to the present invention.

Preferred Growth Hormones or Secretagogues

Preferred growth hormones are methionylated human growth hormone (Met-hGH) and human growth hormone (hGH), human growth hormone being most preferred. The preferred growth hormone secretagogues are 5-amino-5-methyl-hex-2-enoic acid N-methyl-N-((1R)-1-(methyl-((1R)-1-(methylcarbamoyl)-2-phenylethyl)carbamoyl)-2-(naphthalen-2-yl)ethyl)amide with the formula I:

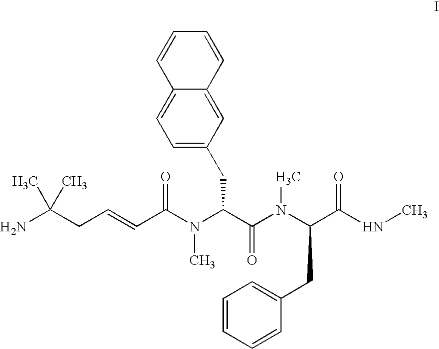

and H-Aib-His-D-2Nal-D-Phe-Lys-NH2 with the formula II:

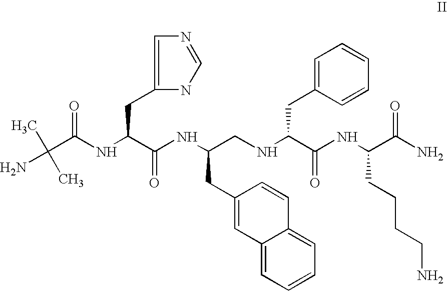

and N-[1(R)-[(1,2-Dihydro-1-methanesulfonylspiro[3H-indole-3,4′-piperidin]-1′-yl)carbonyl]-2-(phenylethyloxy)ethyl]-2-amino-2-methylpropanamide mesylate with the formula III:

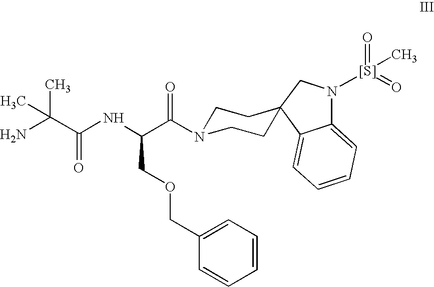

where 5-amino-5-methyl-hex-2-enoic acid N-methyl-N-((1R)-1-(methyl-((1R)-1-(methylcarbamoyl)-2-phenylethyl)carbamoyl)-2-(naphthalen-2-yl)ethyl)amide (formula I) is most preferred.

Abbreviations

H-Aib=H-amino-isobutyric acid

D-2Nal=D-2-naphthylalanine

D-Phe=D-phenylalanine

Pharmaceutical Administration

The regimen for any patient to be treated with growth hormone or growth hormone secretagogues as mentioned herein should be determined by those skilled in the art. The daily dose to be administered in therapy can be determined by a physician and will depend on the particular compound employed, on the route of administration and on the age and the condition of the patient. A convenient daily dosage is suitably from about 6 μg/kg/day to about 720 μg/kg/day, preferably from about 6 μg/kg/day to about 350 μg/kg/day, more preferred from about 30 μg/kg/day to about 200 μg/kg/day, most preferred from about 50 μg/kg/day to about 150 μg/kg/day.

The growth hormone or growth hormone secretagogue should be administered during the distraction period and may further be administered during some or all of the healing period (following the distraction period) until bone quality is satisfying. The administration period would normally be in the range of from about 5 weeks to about 200 weeks, preferably from about 20 weeks to about 100 weeks.

The route of administration may be any route which effectively transports the active compound to the appropriate or desired site of action, such as oral, nasal, vaginal, rectal, sublingual, transdermal or parenteral (e.g., intramuscular, intraperitonal, intravenous or subcutaneous injection, or implant) as well as pulmonary inhalation. The growth hormone or growth hormone secretagogue can be formulated in dosage forms appropriate for each route of administration. The compositions or dosage forms may appear in conventional forms, for example capsules, tablets, aerosols, solutions, suspensions or topical applications.

However, the protein nature of growth hormone itself makes anything but parenteral administration non-viable. Furthermore, other directly acting natural secretagogues, e.g., GHRH and PACAP, are longer polypeptides for which reason oral administration is not viable. The growth hormone or growth hormone secretagogue may be administered subcutaneously, intravenously or intramuscularly or it may be administered by continuous or pulsatile infusion to the bone surfaces to be healed. According to a preferred aspect of the invention, the growth hormone is administered subcutaneously.

Other secretagogues, e.g. arginine, L-3,4-dihydroxyphenylalanine (L-Dopa), muscarinic receptor agonists, 5-amino-5-methyl-hex-2-enoic acid N-methyl-N-((1R)-1-(methyl-((1R)-1-(methylcarbamoyl)-2-phenylethyl)carbamoyl)-2-(naphthalen-2-yl)ethyl)amide (formula I), H-Aib-His-D-2Nal-D-Phe-Lys-NH2 (formula II), and N-[1(R)-[(1,2-Dihydro-1-methanesulfonylspiro[3H-indole-3,4′-piperidin]-1′-yl)carbonyl]-2-(phenylethyloxy)ethyl]-2-amino-2-methylpropanamide mesylate (formula III) are smaller molecules, which may be viable for oral, vaginal, rectal, nasal, pulmonal or transdermal administration. The oral route is preferred. Such compounds may optionally be on a pharmaceutically acceptable salt form such as the pharmaceutically acceptable acid addition salts of an inorganic or organic acid such as hydrochloric, hydrobromic, sulfuric, acetic, phosphoric, lactic, maleic, phthalic, citric, glutaric, gluconic, methanesulfonic, salicylic, succinic, tartaric, toluenesulfonic, trifluoracetic, sulfamic or fumaric acid, or in the form of an alkali metal or alkaline earth metal or lower alkylammonium salt. Such salt forms are believed to exhibit approximately the same order of activity as the free base forms.

Solid dosage forms for oral administration include capsules, tablets, pills, powders and granules. In such solid dosage forms, the active compound is admixed with at least one inert pharmaceutically acceptable carrier such as sucrose, lactose, or starch. Such dosage forms can also comprise, as in normal practice, additional substances other than inert diluents, e.g., lubricating agents such as magnesium stearate. In the case of capsules, tablets and pills, the dosage forms may also comprise buffering agents. Tablets and pills can additionally be prepared with enteric coatings.

Liquid dosage forms for oral administration include pharmaceutically acceptable emulsions, solutions, suspensions, syyrups, the elixirs containing inert diluents commonly used in the art, such as water. Besides such inert diluents, compositions can also include adjuvants, such as wetting agents, emulsifying and suspending agents, and sweetening, flavouring, and perfuming agents.

Preparations for parenteral administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized by, for example, filtration through a bacteria-retaining filter, by incorporating sterilizing agents into the compositions, by irradiating the compositions, or by heating the compositions. They can also be manufactured in the form of sterile solid compositions which can be dissolved in sterile water, or some other sterile injectable medium prior to or immediately before use.

Compositions for rectal or vaginal administration are preferrably suppositories which may contain, in addition to the active substance, excipients such as cocoa butter or a suppository wax. Compositions for nasal or sublingual administration are also prepared with standard excipients well known in the art. Conventional techniques for preparing pharmaceutical compositions usable according to the present invention are, for example, described in Remington's Pharmaceutical Sciences, 1985. The compositions may appear in conventional forms, for example capsules, tablets, aerosols, solutions, suspensions or topical applications.

The pharmaceutical carrier or diluent employed may be a conventional solid or liquid carrier. Examples of solid carriers are lactose, terra alba, sucrose, cyclodextrin, talc, gelatin, agar, pectin, acacia, magnesium stearate, stearic acid or lower alkyl ethers of cellulose. Examples of liquid carriers are syrup, peanut oil, olive oil, phospholipids, fatty acids, fatty acid amines, polyoxyethylene or water.

Similarly, the carrier or diluent may include any sustained release material known in the art, such as glyceryl monostearate or glyceryl distearate, alone or mixed with a wax.

If a solid carrier is used for oral administration, the preparation may be tabletted, placed in a hard gelatin capsule in powder or pellet form or it can be in the form of a troche or lozenge. The amount of solid carrier will vary widely but will usually be from about 25 mg to about 1 g. If a liquid carrier is used, the preparation may be in the form of a syrup, emulsion, soft gelatin capsule or sterile injectable liquid such as an aqueous or non-aqueous liquid suspension or solution.

A typical tablet which may be prepared by conventional tabletting techniques may contain:

| Core: | | |
|---|---|---|
| Active compound (as free compound or salt thereof) | 100 | mg |
| Colloidal silicon dioxide (Aerosil) | 1.5 | mg |
| Cellulose, microcryst. (Avicel) | 70 | mg |
| Modified cellulose gum (Ac-Di-Sol) | 7.5 | mg |
| Magnesium stearate | | |
| Coating: | | |
| HPMC approx. | 9 | mg |
| *Mywacett 9-40 T approx. | 0.9 | mg |

*Acylated monoglyceride used as plasticizer for film coating.

For nasal administration, the preparation may contain a growth hormone secretagogue dissolved or suspended in a liquid carrier, in particular an aqueous carrier, for aerosol application. The carrier may contain additives such as solubilizing agents, e.g. propylene glycol, surfactants, absorption enhancers such as lecithin (phosphatidylcholine) or cyclodextrin, or preservatives such as parabenes.

The present invention is further illustrated by the following examples which are not in any way intended to limit the scope of the invention as claimed.

### EXAMPLES

### Example 1

Preparation of 5-amino-5-methyl-hex-2-enoic acid N-methyl-N-((1R)-1-(methyl-((1R)-1-(methylcarbamoyl)-2-phenylethyl)carbamoyl)-2-(naphthalen-2-yl)ethyl)amide with the formula:

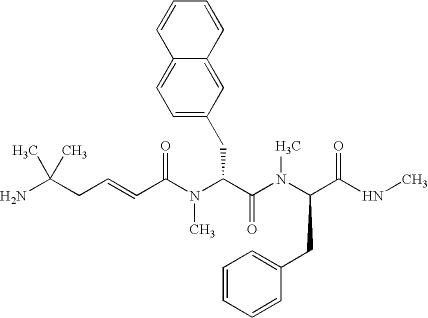

3-Hydroxy-1,1-dimethylpropylcarbamic acid tert-butyl ester:

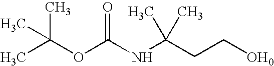

Step A: At 0° C., ethyl chloroformate (1.10 mL, 11.5 mmol) was given dropwise to a solution of 3-tert-butoxycarbonylamino-3-methylbutanoic acid (2.50 g, 11.5 mmol) and triethylamine (1.92 mL, 13.8 mmol) in tetrahydrofuran (10 mL). The solution was stirred for 40 min at 0° C. The formed precipitate was filtered off and washed with tetrahydrofuran (20 mL). The liquid was immediately cooled to 0° C. A 2M solution of lithium boronhydride in tetrahydrofuran (14.4 mL, 28.8 mmol) was added dropwise. The solution was stirred at 0° C. for 2 h, and then warmed to room temperature, over a period of 4 h. It was cooled to 0° C. Methanol (5 mL) was added carefully. 1N Hydrochloric acid (100 mL) was added. The solution was extracted with ethyl acetate (2×100 mL, 3×50 mL). The combined organic layers were washed with saturated sodium hydrogen carbonate solution (100 mL) and dried over magnesium sulfate. The solvent was removed in vacuo. The crude product was chromatographed on silica (110 g) with ethyl acetate/heptane 1:2 to give 1.84 g of 3-hydroxy-1,1-dimethylpropylcarbamic acid tert-butyl ester.

¹H-NMR (CDCl₃): d 1.33 (s, 6H); 1.44 (s, 9H); 1.88 (t, 2H); 1.94 (br, 1 H); 3.75 (q, 2H); 4.98 (br, 1H).

3-(tert-Butoxycarbonylamino)-3-methylbutanal:

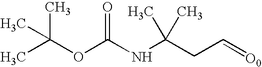

Step B: DMSO (1.22 mL, 17.2 mmol) was added to a solution of oxalyl chloride (1.1 mL, 12.9 mmol) at −78° C. in dichloromethane (15 mL). The mixture was stirred for 15 min at −78° C. A solution of 3-hydroxy-1,1-dimethylpropylcarbamic acid tert-butyl ester (1.75 g, 8.6 mmol) in dichloromethane (10 mL) was added dropwise over a period of 15 min. The solution was stirred at −78° C. for another 15 min. Triethylamine (6.0 mL, 43 mmol) was added. The solution was stirred at −78° C. for 5 min and then warmed to room temperature. The solution was diluted with dichloromethane (100 mL) and extracted with 1N hydrochloric acid (100 mL). The aqueous phase was extracted with dichloromethane (50 mL). The combined organic layers were washed with saturated sodium hydrogen carbonate solution (100 mL) and dried over magnesium sulfate. The solvent was removed in vacuo. The crude product was purified by column chromatography on silica (140 g) with ethyl acetate/heptane (1:3) to give 1.10 g of 3-(tert-butoxycarbonylamino)-3-methylbutanal. MHz-¹H-NMR (CDCl₃): d 1.39 (s, 6H); 1.45 (s, 9H); 2.85 (d, 2H); 4.73 (br. 1H); 9.80 (t, 1H).

Ethyl (2E)-5-(tert-Butoxycarbonylamino)-5-methylhex-2-enoate:

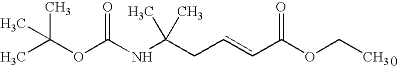

Step C: Triethylphoshonoacetate (1.96 mL, 9.8 mmol) was dissolved in tetrahydrofuran (30 mL). Potassium tert-butoxide (1.10 g, 9.8 mmol) was added. The solution was stirred for 40 min at room temperature. A solution of 3-(tert-butoxycarbonylamino)-3-methylbutanal (1.10 g, 5.5 mmol) in Tetrahydrofuran (6 mL) was added. The solution was stirred at room temperature. for 75 min. It was diluted with ethyl acetate (100 mL) and 1N hydrochloric acid (100 mL). The phases were separated. The aqueous phase was extracted with ethyl acetate (2×50 mL). The combined organic phases were washed with saturated sodium hydrogen carbonate solution (60 mL) and dried over magnesium sulfate. The solvent was removed in vacuo. The crude product was purified by column chromatography on silica (90 g) with ethyl acetate/hepatane (1:4) to give 1.27 g of ethyl (2E)-5-(tert-butoxycarbonylamino)-5-methylhex-2-enoate.

¹H-NMR (CDCl₃): d 1.30 (s, 6H); 1.30 (t, 3H); 1.46 (s, 9H); 2.62 (d, 2H); 4.27 (q, 2H); 4.42 (br, 1H); 5.88 (d, 1H); 6.94 (td, 1H).

(2E)-5-(tert-Butoxycarbonylamino)-5-methylhex-2-enoic acid:

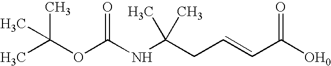

Step D: Ethyl (2E)-5-(tert-butoxycarbonylamino)-5-methylhex-2-enoate (1.233 g, 4.54 mmol) was dissolved in dioxane (20 mL). Lithium hydroxide (0.120 g, 5.00 mmol) was added as a solid. Water (10 mL) was added, until a clear solution was reached. The solution was stirred 16 h at room temperature. The solution was diluted with water (70 mL) and was extracted with tert-butyl methyl ether (2×100 mL). The aqueous phase was acidified with 1N sodium hydrogensulfate solution (pH=1) and was extracted with tert-butylmethylether (3×70 mL). The organic phases were combined and dried over magnesium sulfate. The solvent was removed in vacuo to give 1.05 g of (2E)-5-(tert-butoxycarbonylamino)-5-methylhex-2-enoic acid. The crude product was used for further syntheses.

¹H-NMR (DMSO d₆): d 1.15 (s, 6H); 1.35 (s, 9H); 2.53 (d, 2H); 5.75 (d, 1H); 6.57 (br, 1H); 6.75 (td, 1H); 12.15 (s, 1H).

N-Methyl-N-((R)-1-(methylcarbamoyl)-2-phenylethyl)carbamic acid tert-butyl ester:

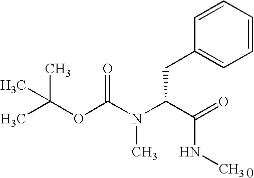

Step E: N-Tert-butoxycarbonyl-N-methyl-D-phenylalanine (1.22 g, 4.4 mmol), 1-hydroxybenzotriazole hydrate(0.59 g, 4.4 mmol) and 1-ethyl-3-(3-dimethylaminopropyl)carbodiimid hydrochloride (0.88 g, 4.6 mmol) were dissolved in N,N-dimethylformamide (25 mL) and stirred for 30 min. Methylamine (0.51 g of a 40% solution in methanol, 6.6 mmol) was added and the mixture was stirred overnight. Methylene chloride (80 mL) and water (100 mL) were added and the phases were separated. The organic phase was washed with sodium hydroxide (20 mL, 1N), sodium hydrogensulfate (50 mL, 10%) and water (50 mL). The organic phase was dried (magnesium sulfate) and the solvent removed in vacuo to afford 1.39 g of N-methyl-N-((R)1-(methylcarbamoyl)-2-phenylethyl)carbamic acid tert-butyl ester.

¹H-NMR (CDCl₃): d 1.25, 1.35 (two s (br), 9H); 2.73-2.94 (m, 7H); 3.30-3.50 (m, 1H); 4.68, 4.90 (two m, 1H); 5.90, 6.12 (two s (br); 1H); 7.12-7.25 (m, 5H).

(R)-N-Methyl-2-methylamino-3-phenylpropionamide:

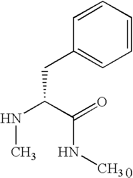

Step F: N-Methyl-N-((R)1-(methylcarbamoyl)-2-phenylethyl)carbamic acid tert-butyl ester (1.39 g, 7.23 mmol) was dissolved in a mixture of trifluoroacetic acid (5 mL) and methylene chloride (10 mL) and stirred for 45 min. The volatiles were removed in vacuo and the residue was stirred with a mixture of ethyl acetate (100 mL) and water (100 mL). Sodium hydrogen carbonate (50 mL, saturated) was added and the phases were separated. The organic phase was dried (magnesium sulfate) and the solvent removed in vacuo to afford 330 mg of (R)-N-methyl-2-methylamino-3-phenylpropionamide.

¹H-NMR (CDCl₃): d 2.1 (s(br), 3H); 2.32 (s, 3H); 2.77 (dd, 1H); 2.81 (two s, 3H); 3.21 (dd, 1H); 3.32 (dd, 1H); 7.12 (s(br), 1H); 7.20-7.34 (m, 5H).

N-Methyl-N-{(1R)-1-(N-methyl-N-((1R)-1-(methylcarbamoyl)-2-phenylethyl)carbamoyl)-2-(2-naphthyl)ethyl}carbamic acid tert-butyl ester:

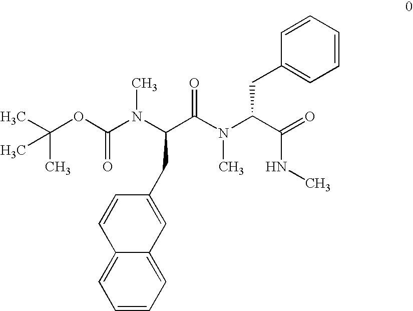

Step G: (R)-Tert-butoxycarbonyl-N-methylamino-3-(2-naphthyl)propionic acid (548 mg, 1.66 mmol) was dissolved in methylene chloride (5 mL); 1-hydroxy-7-azabenzotriazole (227 mg, 1.66 mmol) was added along with N,N-dimethylformamide (2 mL). 1-Ethyl-3-(3-dimethylaminopropyl)carbodiimide hydrochloride (351 mg, 1.83 mmol) was added and the solution was stirred for 15 min. (R)-N-Methyl-2-methylamino-3-phenylpropionamide (320 mg, 1.66 mmol) dissolved in methylene chloride (4 mL) and diisopropylethylamine (0.28 mL, 1.66 mmol) were added and the mixture was stirred overnight. Methylene chloride (50 mL) was added and the organic phase was washed with water (100 mL), sodium hydrogensulfate (50 mL, 5%) and sodium hydrogen carbonate (50 mL, saturated). The organic phase was dried (magnesium sulfate) and the solvent removed in vacuo. The residue was chromatographed (silica, 2×45 cm) using ethylacetate/methylene chloride (1:1) to afford 604 mg of N-methyl-N-{(1R)-1-(N-methyl-N-((1R)-1-(methylcarbamoyl)-2-phenylethyl)carbamoyl)-2-(2-naphth-yl)ethyl}carbamic acid tert-butyl ester.

¹H-NMR (CDCl₃): d 1.05, 1.31, 1.56 (three s, 9H); 2.28-3.37 (several m, 13H); 5.04, 5.17, 5.29, 5.48 (four dd, 2H); 7.05-7.79 (m, 12H).

(2R)-N-Methyl-2-methylamino-N-((1R)-1-(methylcarbamoyl)-2-phenylethyl)-3-(2-naphthyl)propionamide:

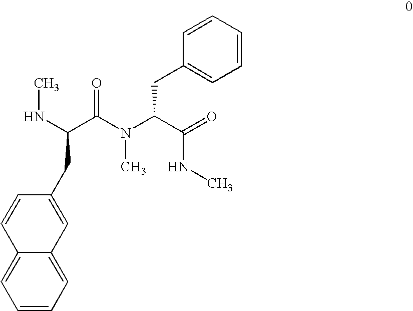

Step H: N-Methyl-N-{(1R)-1-(N-methyl-N-((1R)-1-(methylcarbamoyl)-2-phenylethyl)carbamoyl)-2-(2-naphthyl)ethyl}carbamic acid tert-butyl ester (600 mg, 1.19 mmol) was stirred in trifluoroacetic acid/methylene chloride (1:1, 5 mL) for 10 min and the volatiles were removed in vacuo. The residue was stripped with diethylether (2×5 mL) and dissolved in methanol (2 mL) and mixed with sodium hydrogen carbonate (10 mL) and ethylacetate (15 mL). The organic phase was separated and dried (magnesium sulfate) to afford 420 mg of (2R)-N-methyl-2-methylamino-N-((1R)-1-(methylcarbamoyl)-2-phenylethyl)-3-(2-naphthyl)propionamide.

¹H-NMR (CDCl₃): (selected values) d 1.69 (s, 3H); 2.08 (d, 3H); 2.54 (s, 3H); 2.76 (dd, 1H); 2.92 (dd, 1H); 3.12 (dd, 1H), 3.31 (dd, 1H); 3.72 (dd, 1H), 4.95 (q (br), 1H); 5.50 (dd, 1H).

((3E)-1,1-Dimethyl-4-(N-methyl-N-((1R)-1-(N-methyl-N-((1R)-1-(methylcarbamoyl)-2-phenylethyl)carbamoyl)-2-(2-naphthyl)ethyl)carbamoyl)but-3-enyl)carbamic acid tert-butyl ester:

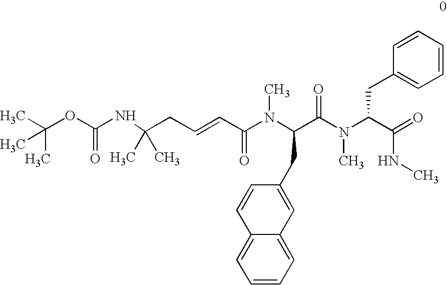

Step I: (2E)-5-(tert-Butyloxycarbonylamino)-5-methylhex-2-enoic acid (200 mg, 0.82 mmol), 1-hydroxy-7-azabenzotriazole (112 mg, 0.82 mmol) and 1-ethyl-3-(3-dimethylaminopropyl)-carbodiimide hydrochloride (173 mg, 0.90 mmol) were dissolved in a mixture of methylene chloride (10 mL) and N,N-dimethylformamide (1 mL) and strirred for 15 min. N-Methyl-2-methylamino-N-((1R)-1-(methylcarbamoyl)-2-phenylethyl)-3-(2-naphthyl)propionamide (332 mg, 0.82 mol) dissolved in methylene chloride (5 mL) and diisopropylethylamine (0.14 mL) were added and the mixture was stirred overnight under nitrogen atmosphere. The mixture was diluted with methylene chloride (50 mL), washed with water (50 mL), sodium hydrogen carbonate (30 mL, saturated), and sodium hydrogensulfate (30 mL, 5%). The phases were separated and the organic phase was dried with magnesium sulfate and evaporated in vacuo. The residue was chromatographed (silica, 2×40 cm) to afford 450 mg of ((3E)-1,1-dimethyl-4-(N-methyl-N-((1R)-1-(N-methyl-N-((1R)-1-(methylcarbamoyl)-2-phenylethyl)carbamoyl)-2-(2-naphthyl)ethyl)carbamoyl)but-3-enyl)-carbamic acid tert-butyl ester.

¹H-NMR (CDCl₃): (selected values) d 1.20, 1.22, 1.24, 1.30, 1.41, 1.55 (six s, 15H), 4.30, 4.40 (two s (br), 1H); 5.08, 5.18, 5.32, 5.60, 5.87 (five dd, 2H); 6.05 (dd, 1H); 6.75 (m, 1H).

Step J:

((3E)-1,1-Dimethyl-4-(methyl-((1R)-1-(methyl-((1R)-1-(methylcarbamoyl)-2-phenylethyl)carbamoyl)-2-(2-naphthyl)ethyl)carbamoyl)but-3-enyl)carbamic acid tert-butyl ester (403 mg, 0.63 mmol) was stirred in a mixture of trifluroacetic acdi (4 mL) and methylene chloride (4 mL) for 10 min. The volatiles were removed in vacuo and the crude product was chromatographed on silica (400 g) using a mixture of methylene chloride, ethanol and ammonia (25% in water) (80/18/2) as eluent. The isolated product was dissolved in 3M hydrochloric acid in ethyl acetate and evaporated, then redissolved in methylene chloride and evaporated twice to afford 140 mg of the title compound.

¹H-NMR (CDCl₃): d 1.05, 1.10, 1.15, 1.16 (four s, 6H); 2.07 (s (br); 3H); 5.12, 5.32, 5.40, 5.60, 5.91 (five dd, 2H); 6.05, 6.14 (two d, 1H); 6.80 (m, 1H)

HPLC: Rₜ=29.02 min (Method A1)

ESMS: m/z=529 (100%)(M+H)⁺

Example 2

In comparison to rats and rabbits, the micropig animal model exhibits superior similarities with humans in terms of endocrinological cycles and fracture repair processes. The bone healing was monitored continously by the in-vivo initial torsional stiffness measurements, as opposed to one-time destructive mechanical testing in previous studies.

Mini Pigs

30 female Yucatan mini-pigs were obtained from Charles River (Saint Aubin lés Elbeuf, France).

Anesthesia and Surgery

Prior to surgery, the pigs had Stresnil® (4 mg/kg) and Atropin (0.05 mg/kg) i.m. for sedation. Using a port-catheter system, which had been implanted in a previous operation into the external jugular vein (see below), the pigs got 6-8 ml Thiopental i.v. (5 mg/kg). The pigs were intubated, using a tubus of 7.5-7.0 mm diameter. Following intubation, the pigs were given 2 ml Pancuronium (0.13 mg/kg) for muscular relaxation. During surgery, the pigs were given artificial respiration and general anesthesia were maintained with intravenous Thiopental and Fentanyl.

The tibial bone was inspected by fluoroscopy. The insertion places for the 4.5 mm Titanium Schanz' screws were defined 3 cm proximally and distally of the planned osteotomy in the middle of the tibia. The fibula was osteotomized with removal of a 5 mm piece. The fixator frame was fixated with alignment to the mid-shaft axis. The tibia was osteotomized horizontally. Documentation and proof of axis-alignment was done by X-ray analysis.

Measurements

Torsional Stiffness

A modified external ring fixator and stiffness measurement apparatus was designed to measure torsional stiffness during the healing period. This fixator allows torque movement of the bone (axial direction) for measurement. The measurement apparatus consist of a load cell (HBM, EF7A H3/57K/125 lbs, Hottinger Baldwin Messtechnik) and a displacement element (HBM, WSF/20 mm, ID-No. 1094/1, Hottinger Baldwin Messtechnik), which are connected to a measurement-amplifier (HBM, MGC, Hottinger Baldwin Messtechnik) and a personal computer (HBM, MGC, Hottinger Baldwin Messtechnik).

The first measurement was taken immediately post-operative on day 0. After the distraction period (day 15), stiffness measurements were performed on days 15, 19, 21, 24, and 28. For measurements, the animal was sedated and separated in a hammock for unsupported limb-hanging. The protection cover and the fixator bolts were removed and the load-displacement unit placed side-by-side to the fixator. Four cycles of motion were applied and resulting forces and displacement recorded. In order not to destroy woven bone, the measurements were limited to angulations of 15 to 20°. In order not to refracture, the last days measurements were limited to torques about 10 Nm.

Conventional X-ray

X-rays of the distracted limb were obtained every testing day (same days as stiffness meaurements were performed) to control alignment and fixation. The distance between focus and X-ray film was 115 cm. Two different exposure settings were performed: 1) 66 KV and 2.5 mAs; 2) 55 KV and 5.0 mAs. The films were digitized via an image analysis system (Pace-System Diagnostix 2048), data were stored on WORM. The evaluation of bone densities were accustomed with Diagnostix 2048 and internal software. A second analysis was performed using NIH-image software on Macintosh-PC. Standardisation of the X-ray was established using a 10 chamber aluminium phantom and a 6 chamber hydroxiapatite phanthom.

Digital X-ray

The classic X-ray method was repeated using Siemens Digiscan diagnostics.

Ultrasound

A 7.5 MHz transducer (PICKER CS 9500) with gel pad for improved ultrasound wave transmission was used for the measurement of distance of sound transmission, greyscale density of cortical bone, distraction tissue, and width of distraction gap. The measurements was conducted same days as stiffness measurements were performed. For each animal, a transducer holder has been shaped to guarantee repeatable positions of the transducer with respect to the distraction gap. Three images per examination was taken. The images was evaluated using NIH-software on a Macintosh computer.

Blood Samples

Four samples per hour was taken during a 6-hour period from central veneous catheterization through pig ear veins to measure basal GH levels. Port-catheter systems was implantated into each pig thereby penetrating the vena jugularis extema. 14 blood samples were taken from each pig, sample I was taken before administration of GH or placebo. Sample 2 was taken at day 0 (initial surgery and first day of GH- or placebo application). The following samples was taken the same days as stiffness measurements were performed until sacrifice. Blood sampling was carried out with 2 ml blood wasted, then taking 3 samples of 5.5 ml serum, and 2 samples of 5.5 ml heparin blood. The samples were centrifuged (10 minutes at 3000 rpm). IGFI, free IGFI, IGF binding proreins, total and free T3 and T4, and bone specific alkaline phosphatase were measured.

QCT Scan

All animals were examined using Computed Tomography (CT) CT scans of the right limb was taken horizontally with 3 mm scans at 3 mm intervals. A solid hydroxyapatite phantom was included.

Mechanical Torsion Testing

After sacrifice, the right and left limb were dissected. The soft tissue around the joints was removed, and the ends of the tibia were casted in Beracryl. The constructs were placed in a materials testing machine (Zwick, Ulm, Germany), and torsional moments were applied. Resulting displacements and torsional moments were recorded. The test was conducted with a speed of 30° per minute until failure. The system automatically terminated the test at a torque of 20%.

Histology

30 days before sacrifice, a calcein green was administered i.v. at a dosage of 15 mg/kg body weight. 20 days before necropsy, tetracycline at a dosage of 25 mg/kg body weight was administered i.v. 5 days before sacrifice, xylenorange (90 mg/kg body weight) was administered i.v. Both tibiae, one lumbar spine body, a pelvic section, and the left ninth rib were harvested. After terminal stiffness measurements, fixation of bone according to Delling (one part Formalin solution 37% (acid free) and 9 parts phosphate-buffer solution pH 7.0) for preservation of perfect structure integrity was carried out. Subsequently, preparations of 3 mm and 2 mm sections (frontal: tibiae, lumbar spine body; vertical: tibiae, pelvis rib) with the Exact grinding system were carried out. The 2 mm specimen was infiltrated using a combination of alcohol and Technovit 7200 VLC. The embedding in Technovit 7200 VLC comprised two steps: 1) low light intensity, temperature 40° C.; 2) high light intensity, the total polymerisation lasted 6 hours. The specimen was grinded unstained down to a thickness of 30 μm, suitable for fluorescence microscopy.

The 3 mm section was embedded in Kulzer Technovit 9100 methylmetacrylate and polymerized in a drying stove at 37° C. Mid-frontal 5 μm-sections were produced. After the usual histological preparation processes, the following stainings were applied. Goldner trichrome: This stain is suitable for differentiation of osteoid and mineralized bony structures and additionally for differentiation of osteoclasts (large, irregularly shaped, foamy and slightly metachromatic cytoplasma containing one or more nuclei, residing with Howship's lacunae), osteoblasts (mononuclear, basophilic, cuboidal, prominent Golgi apparatus, contact with osteoi) and osteocytes (small, within bone, surrounded by canaliculi). Von Kossa stain combined with safranine-O: A fair contrast between mineralized bone (dark brown/black), cartilage (dark red) and connective tissue (light red) is achieved. Astra-blue: This stain allows the discrimination of calcified cartilage (dark blue) as a hint for active chondral ossification. Sirius red: With a suitable polarisation and lambda filter, the difference between woven bone and lamellar bone is demonstrated.

The static and the dynamic parameters of the bone and callus were measured with an image analysis system (Leitz Quantimed). The microscopic image of the interesting site was digitized by a 3-chip-CCD camera (Sony) and loaded into the RAM of an image processor of the image analysis work station. Semi-automatic processing of the image over-shading, normalisation, enhancement and binarisation provided isolation of the interesting structure and the next step for the pixel-based measurement of this structure.

Acceleration of Callus Distraction with Recombinant Growth Hormone in Mini Pigs

The final bone torsional strength of a mini-pig group treated with growth hormone was investigated relative to a control group.

Methods

30 mature female Yucatan mini-pigs were equally distributed into two treatment groups. Mini-pigs in the study group received a daily injection of recombinant porcine growth hormone (r-pGH: 100 μg/kg), the mini-pigs in the control group received sodium chloride as placebo. The left tibiae and fibulae were osteotomized and stabilized with an external fixator. Full weight bearing was permitted. The legs were distracted twice daily at 2 mm/day for 10 days and then allowed to consolidate for 10 days. After sacrifice, torsional moments were applied to the distracted and the contralateral tibia in a material testing machine and the maximum torsional moment was determined. Four animals were removed from the study due to bone infection or surgery failure and data of 2 animals were lost due to test machine failure. A t-test was used to determine differences in maximum torsional moments between the treatment groups.

Results

The tibiae of the GH-treated micropig group exhibited 131% higher maximum torsional moment than those of the placebo group (GH: 19.5±7.8 Nm, Placebo 8.45±5.4 Nm, p=0.001). In comparison to the intact contralateral tibia, the GH group and the control group reached 64±22.4% and 26±12.9% maximum torsional moment (p<0.001).

Discussion

The results demonstrate that GH greatly increases bone regenerate torsional strength at sacrifice. To our knowledge it is the first study using homologous GH in a large animal model, while other studies used bovine or human GH in rats or rabbits. In this study, we examined bone regenerate derived from distraction osteogenesis, a method of relatively standardized bone formation, as opposed to the non- standardized fracture creation of other studies. The results demonstrate that systemic administration of homologous GH accelerates consolidation of bone regenerate in callus distraction.

1. A method of promoting bone formation in an animal simultaneous with callus distraction, comprising administering to an animal in need thereof an effective amount of a growth hormone or a growth hormone secretagogue.

2. The method of claim 1, wherein the animal is a human being.

3. The method of claim 1, wherein the growth hormone is human growth hormone.

4. The method of claim 1, wherein the growth hormone secretagogue is selected from the group consisting of:
5-amino-5methyl-hex-2-enoic acid N-methyl-N-((1R)-1-(methyl-((1R)-1-(methylcarbamoyl)-2-phenylethyl)carbamoyl)-2-(naphthalen-2-yl)ethyl)amide of formula I: 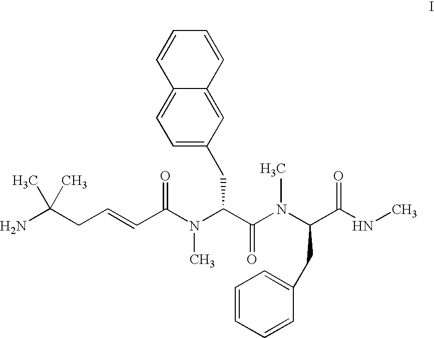
H-Aib-His-D-2Nal-D-Phe-Lys-NH2 of formula II: 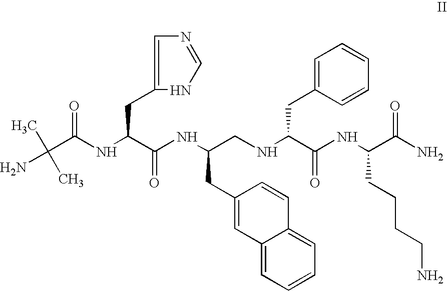
and N-[1(R)-[(1,2-Dihydro-1-methanesulfonylspiro[3H-indole-3,4′-piperidin]-1′-yl)carbonyl]-2-(phenylmethyloxy)ethyl]-2-amino-2-methylpropanamide mesylate of formula III:

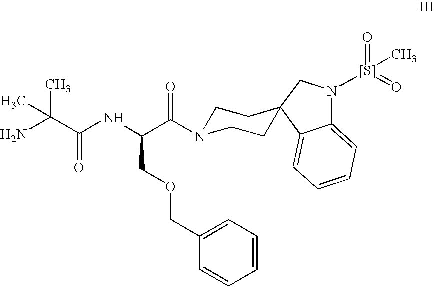

5. A method of enhancing the healing of bone fractures in an animal subjected to distraction osteogenesis, the method comprising administering, to the animal in need thereof, a growth hormone or a growth hormone secretagogue in an amount sufficient to provide bone formation simultaneous with the distraction procedure.

6. The method of claim 5, wherein the animal is a human being.

7. The method of claim 5, wherein the growth hormone is human growth hormone.

8. The method of claim 5, wherein the growth hormone secretagogue is selected from the group consisting of:
5-amino-5-methyl-hex-2-enoic acid N-methyl-N-((1R)-1-(methyl-((1R)-1-(methylcarbamoyl)-2-phenylethyl)carbamoyl)-2-(naphthalen-2-yl)ethyl)amide (formula I);
H-Aib-His-D-2Nal-D-Phe-Lys-NH2 (formula II); and
N-[1(R)-[(1,2-Dihydro-1-methanesulfonylspiro[3H-indole-3,4′-piperidin]-1′-yl)carbonyl]-2-(phenylethyloxy)ethyl]-2-amino-2-methylpropanamide mesylate (formula III).

9. A method of treating patients suffering from fractured bones, and post-traumatic and idiopathic deformities of extremities by distraction osteogenesis, comprising administering an effective amount of a growth hormone or a growth hormone secretagogue to a patient in need of such a treatment in conjunction with the distraction procedure.

10. The method of claim 9, wherein the patient is a human being.

11. The method of claim 9, wherein the growth hormone is human growth hormone.

12. The method of claim 9, wherein the growth hormone secretagogue is selected from the group consisting of:
5-amino-5-methyl-hex-2-enoic acid N-methyl-N-((1R)-1-(methyl-((1R)-1-(methylcarbamoyl)-2-phenylethyl)carbamoyl)-2-(naphthalen-2-yl)ethyl)amide (formula I);
H-Aib-His-D-2Nal-D-Phe-Lys-NH2 (formula II); and
N-[1(R)-[(1,2-Dihydro-1-methanesulfonylspiro[3H-indole-3,4′-piperidin]-1′-yl)carbonyl]-2-(phenylethyloxy)ethyl]-2-amino-2-methylpropanamide mesylate (formula III).
